SEQUENCE LISTING (28 sequences)

SEQ ID NO: 1 - protein, 8 aa, Hippocampus trimaculatus
GTEDELDK

SEQ ID NO: 2 - protein, 6 aa, Hippocampus trimaculatus
FLHDVQ

SEQ ID NO: 3 - DNA, 25 bp, Artificial Sequence
  SYNTHESIZED PRIMER
cccttccgaagtttctggcagcagc

SEQ ID NO: 4 - DNA, 24 bp, Artificial Sequence
  SYNTHESIZED PRIMER
ggctgtcaggcctcgtggctttgg

SEQ ID NO: 5 - DNA, 27 bp, Artificial Sequence
  SYNTHESIZED PRIMER
ggggtaccttccagctgtcaaaatctc

SEQ ID NO: 6 - DNA, 25 bp, Artificial Sequence
  SYNTHESIZED PRIMER
gaagatctcgccaggtactcacctg

SEQ ID NO: 7 - DNA, 21 bp, Artificial Sequence
  SYNTHESIZED PRIMER
atgagcacagaaagcatgatc

SEQ ID NO: 8 - DNA, 21 bp, Artificial Sequence
  SYNTHESIZED PRIMER
tacaggcttgtcactcgaatt

SEQ ID NO: 9 - DNA, 25 bp, Artificial Sequence
  SYNTHESIZED PRIMER
atggcaactgttcctgaactcaact

SEQ ID NO: 10 - DNA, 26 bp, Artificial Sequence
  SYNTHESIZED PRIMER
tttcctttcttagatatggacaggac

SEQ ID NO: 11 - DNA, 20 bp, Artificial Sequence
  SYNTHESIZED PRIMER
agttgccttcttgggactga

SEQ ID NO: 12 - DNA, 20 bp, Artificial Sequence
  SYNTHESIZED PRIMER
cagaattgccattgcacaac

SEQ ID NO: 13 - DNA, 20 bp, Artificial Sequence
  SYNTHESIZED PRIMER
gttgggatgaaccagaagga

SEQ ID NO: 14 - DNA, 20 bp, Artificial Sequence
  SYNTHESIZED PRIMER
cttacaatttcccgctctgc

SEQ ID NO: 15 - DNA, 19 bp, Artificial Sequence
  SYNTHESIZED PRIMER
cacaacttctacaacgtgg

SEQ ID NO: 16 - DNA, 19 bp, Artificial Sequence
  SYNTHESIZED PRIMER
gtagatctcggccatgatg

SEQ ID NO: 17 - DNA, 20 bp, Artificial Sequence
  SYNTHESIZED PRIMER
caacagaagctggtggacaa

SEQ ID NO: 18 - DNA, 19 bp, Artificial Sequence
  SYNTHESIZED PRIMER
agatacgacagcattggca

SEQ ID NO: 19 - DNA, 20 bp, Artificial Sequence
  SYNTHESIZED PRIMER
gttgggatgaaccagaagga

SEQ ID NO: 20 - DNA, 20 bp, Artificial Sequence
  SYNTHESIZED PRIMER
cttacaatttcccgctctgc

SEQ ID NO: 21 - DNA, 22 bp, Artificial Sequence
  SYNTHESIZED PRIMER
cgagtgccaaatgaagaggacc

SEQ ID NO: 22 - DNA, 22 bp, Artificial Sequence
  SYNTHESIZED PRIMER
aaacctgagccagaacctgacg

SEQ ID NO: 23 - DNA, 20 bp, Artificial Sequence
  SYNTHESIZED PRIMER
gtactgtgcagccctggaat

SEQ ID NO: 24 - DNA, 20 bp, Artificial Sequence
  SYNTHESIZED PRIMER
tttagaaactgggccacctc

SEQ ID NO: 25 - DNA, 22 bp, Artificial Sequence
  SYNTHESIZED PRIMER
gaatttgagcgtctctgtcgaa

SEQ ID NO: 26 - DNA, 22 bp, Artificial Sequence
  SYNTHESIZED PRIMER
ggttatgccaaatgcacttgag

SEQ ID NO: 27 - DNA, 20 bp, Artificial Sequence
  SYNTHESIZED PRIMER
gttgggatgaaccagaagga

SEQ ID NO: 28 - DNA, 20 bp, Artificial Sequence
  SYNTHESIZED PRIMER
cttacaatttcccgctctgc

(19) United States
(12) Patent Application Publication
(10) Pub. No.: US 2014/0094414 A1
(43) Pub. Date: Apr. 3, 2014

(54) COMPOSITION FOR PREVENTING OR TREATING NEURODEGENERATIVE DISEASE INCLUDING BIOACTIVE PEPTIDE AS EFFECTIVE COMPONENT

(71) Applicant: PUKYONG NATIONAL UNIVERSITY INDUSTRY-UNIVERSITY CO, Busan (KR)

(72) Inventors: Se-Kwon KIM, Busan (KR); Ratih PANGESTUTI, Busan (KR)

(73) Assignee: Pukyong National University Industry-University Cooperation Foundation, Busan (KR)

(21) Appl. No.: 13/665,820

(22) Filed: Oct. 31, 2012

(30) Foreign Application Priority Data
Sep. 28, 2012 (KR) 10-2012-0108505

(51) Int. Cl. C07K 7/06

(52) U.S. Cl. CPC C07K 7/06
USPC 514/17.8; 514/17.7; 514/17.9

(57) ABSTRACT

There is provided a new use of protein hydrolysates and peptide derived from seahorses for preventing or treating a neurodegenerative disease, and more particularly a composition and health functional foods for preventing or treating a neurodegenerative disease including seahorse protein hydrolysates or peptide isolated and purified from the same as an effective component.

### CROSS-REFERENCE TO RELATED APPLICATIONS

This application claims the priority of Korean Patent Application No. 2012-108505 filed on Sep. 28, 2012, in the Korean Intellectual Property Office, the disclosure of which is incorporated herein by reference.

### BACKGROUND OF THE INVENTION

1. Field of the Invention

The present invention relates to new use of protein hydrolysates and peptide derived from seahorses for preventing or treating a neurodegenerative disease, and more particularly a composition and health functional foods for preventing or treating a neurodegenerative disease including seahorse protein hydrolysates or peptide isolated and purified from the same as an effective component.

2. Description of the Related Art

A neurodegenerative disease exhibits a degenerative change in neuronal cells of the central nervous system causing various symptoms, such as damages of movement and sensory functions, and inhibitions of a highly difficult function, such as memory, learning, calculation, and deduction. A representative example of a neurodegenerative disease includes Parkinson's disease, Alzheimer's disease, a memory disorder, and the like. A neurodegenerative disease is characterized by death of neuronal cells due to necrosis or apoptosis which is slowly or suddenly progressed. Accordingly, an understanding about death mechanism of neuronal cells must be taken into consideration in order to develop prevention, control, and treatment methods of diseases in the central nervous system.

The central nervous system is composed of neuronal cells and neuroglial cells. Neuroglial cells are the most distributed cells in brain, which forms 90% in the whole brain cells and 50% in the whole brain. It has been known that the neuroglial cells do not generate nerve impulses in itself, but help neuron to perform its unique functions and have the important function of recovering brain tissues that have been damaged. The neuroglial cells are again constituted of three types, such astrocytes, microglia, and oligodendrocytes.

Among them, the microglia also refers as to microgliacyte and is immune cells presented in the central nervous system (CNS) which forms 5 to 10% in the whole brain cells.

The microglia functions as first defense line in the CNS. The microglia is a main intracellular source of an inflammation mediator in the CNS. It involves nervous inflammations by producing nitric oxide (NO), reactive oxygen species (ROS), pro-inflammatory cytokines, and prostaglandins. The activated microglia performs cytophagocytosis and sytoclasis of microorganisms and cell debris after moving into a damaged nervous tissues area.

However, not roles of the microglia as an inflammatory cell are always beneficial. Recently, it is considered that the un-controlled activation of microglia and the continuous excessive nervous inflammation cause various CNS diseases including neurodegenerative diseases. That is, it has been known that the functionally activated microglia causes a death of neuronal cells by producing and secreting an inflammatory mediating material. Accordingly, the activation of microglia involves various types of neurodegenerative diseases. For example, in a case of Alzheimer's disease, it has been well known that the microglia has the important function of forming senile plagues. It is an important example in that anti-inflammatory drugs such as ibuprofen suppress a formation of senile plagues and prevent dementia.

Meanwhile, seahorses are sea fish belonging to syngnathiformse, pipefishes, and are characterized in that it has a body length of approximately 6 cm to 10.5 cm, marked eye prickles, and very short of base length of a dorsal fin. The long thin proboscis is the same as a head length of back side of eyes, the head is bent almost at a right angle as compared with other fishes, and the body is composed of many cuirasses. The color of body is many colors, especially; pale brown mixed with strong brown and also has small spots or patterns. Seahorses refer also as to water horse, Yongrankja, or a horse's head fish as other name.

Such seahorses do not have toxicity but has warm properties, thereby being effective in loosening knotted blood and boosting energy. Accordingly, they can be also used as drugs. From old times, it is conveyed that they have been used when pregnant women have much pain in childbirth, and they have been widely recognized as a medicine to strengthen virility. Furthermore, it is known that seahorses can treat the fundamental causes by supporting energy, strengthening vitality, improving kidney's functions, and supporting kidney's functions a symptom of peeing in spite of oneself and coughing due to a weak body in the case of man.

According to several studies relating to seahorses, it has been investigated that seahorses exhibit anti-fatigue activity, anti-aging activity, a respiratory disease-treating effectiveness, an effect for improving erectile dysfunction, anti-cancer activity, and the like. According to recent studies, it has been reported that the biopeptide isolated and purified from seahorses has activities in releasing collagen and suppressing MMP.

However, there were no reports of a prevention or treatment effect on a neurodegenerative disease, relating to such seahorses.

Therefore, while looking for a material capable of exhibiting excellent effects for preventing or treating a neurodegenerative disease, and also being harmless to humans as being derived from a natural material, the present inventors noted the peptide isolated and identified from seahorses (Hippocampus trimaculatus), a marine animal and experimented a protection activity of brain cells, activity in alleviating oxidative stress, anti-inflammatory activity, and activity in inhibiting protease caused by the peptide. As a result, the present inventors found that the peptide derived from seahorses can protect brain cells from a nerve toxin and oxidative stress, suppress an expression of inflammation mediator, and increase an expression of anti-inflammation cytokines, thereby having excellent anti-inflammation activity, and also suppress expressions of cathepsin B and cathepsin D involving in a production process of Aβ42, a nerve toxin. Therefore, the present inventors completed the present invention.

### SUMMARY OF THE INVENTION

Accordingly, an object of the present invention is to provide an effective composition for preventing or treating a neurodegenerative disease while being harmless to humans.

Another object of the present invention is to provide an effective health functional food for preventing or improving a neurodegenerative disease while being harmless to humans.

Another object of the present invention is to provide a method of preventing or treating a neurodegenerative disease comprising administering to a subject in need thereof seahorse protein hydrolysates as an effective component.

Another object of the present invention is to provide a method of preventing or treating a neurodegenerative disease comprising administering to a subject in need thereof the composition comprising a peptide having an amino acid sequence set forth in SEQ ID NO: 1 or SEQ ID NO: 2 as an effective component.

In order to achieve the above-described objects of the present invention, the present invention provides a composition for preventing or treating a neurodegenerative disease, the composition including seahorse protein hydrolysates as an effective component.

In addition, the present invention provides a method of preventing or treating a neurodegenerative disease comprising administering to a subject in need thereof seahorse protein hydrolysates as an effective component.

In addition, the present invention provides a method of preventing or treating a neurodegenerative disease comprising administering to a subject in need thereof the composition comprising a peptide having an amino acid sequence set forth in SEQ ID NO: 1 or SEQ ID NO: 2 as an effective component.

According to an embodiment of the present invention, such seahorses may be Hippocampus trimaculatus.

According to an embodiment of the present invention, the protein hydrolysates may be one selected from the group consisting of trypsin hydrolysates of seahorses, alpha-chymotrypsin hydrolysates of seahorses, papain hydrolysates of seahorses, and protease E hydrolysate of seahorses.

In addition, the present invention provides a composition for preventing or treating a neurodegenerative disease, the composition including a peptide having the amino acid sequence set forth in SEQ ID NO: 1 or 2 as an effective component.

According to an embodiment of the present invention, the peptide may be derived from Hippocampus trimaculatus.

According to an embodiment of the present invention, the peptide may have an effect of preventing or treating a neurodegenerative disease through protection activity of brain cells, activity of alleviating oxidative stress, anti-inflammatory activity, and activity of suppressing protease.

According to an embodiment of the present invention, the protection activity of brain cells may be accomplished through a mechanism for suppressing a release of nerve toxin due to an activation of microglia.

According to an embodiment of the present invention, the alleviation of oxidative stress may be accomplished through free radical scavenging activity.

According to an embodiment of the present invention, the anti-inflammatory activity may be accomplished through suppressing expressions of inflammation-related mediating factors such as NO, PGE2, iNOS, COX-2, TNF-α, and IL-1β and increasing expressions of anti-inflammatory cytokines such as TGF-β and IL-4.

According to an embodiment of the present invention, the activity of suppressing protease may be accomplished through suppressing expressions of cathepsin B and cathepsin D involving in a process of producing Aβ42.

According to an embodiment of the present invention, the peptide may be included in a concentration of 10 to 10000 μM in a composition.

According to an embodiment of the present invention, the neurodegenerative disease may be selected from the group consisting of Alzheimer's diseases (AD), Parkinson's diseases (PD), Lou Gehrig's diseases, Huntington's diseases (HD), amyotrophic lateral selerosis (ALS), multiple sclerosis, immune system-damaged brain function abnormalities, progressive nervous diseases, metabolic brain diseases, Niemann-Pick diseases, and dementias caused by ischemic stroke and cerebral hemorrhage.

In addition, the present invention provides a health functional food for preventing or improving a neurodegenerative disease, the health functional food including the amino acid sequence set forth in SEQ ID NO: 1 or SEQ ID NO: 2 as an effective component.

According to an embodiment of the present invention, the peptide may be derived from Hippocampus trimaculatus.

According to an embodiment of the present invention, the peptide may have an effect of preventing or improving a neurodegenerative disease through protection activity of brain cells, activity of alleviating oxidative stress, anti-inflammatory activity, and activity of suppressing protease.

According to an embodiment of the present invention, the neurodegenerative disease may be selected from the group consisting of Alzheimer's diseases (AD), Parkinson's diseases (PD), Lou Gehrig's diseases, Huntington's diseases (HD), amyotrophic lateral selerosis (ALS), multiple sclerosis, immune system-damaged brain function abnormalities, progressive nervous diseases, metabolic brain diseases, Niemann-Pick diseases, and dementias caused by ischemic stroke and cerebral hemorrhage.

According to an embodiment of the present invention, the food may be selected from the group consisting of drinks, meats, chocolates, foods, confectionery, pizzas, ramen, other noodles, rice cakes, gums, candies, ice creams, alcohol drinks, liquors, vitamins, and health supplements.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a representative image of Hippocampus trimaculatus, seahorses, according to the present invention;

FIG. 2 is a graph illustrating PC12 cell viability after treating Aβ42 with various concentrations (0.5, 1, 5, 10, 20 μM) to nerve cells-differentiated PC12 cells by using MTT assay;

FIG. 3 is a graph illustrating a degree of suppressing a nerve toxin after treating H. trimaculatus protein hydrolysates to nerve toxin-induced PC12 cell by using MTT assay;

FIG. 4 is a graph illustrating a degree of suppressing a nerve toxin after treating H. trimaculatus protein hydrolysates with various concentrations (10, 50, 100 μM) to nerve toxin-induced PC12 cell by using MTT assay;

FIG. 5 is a graph illustrating a fractionated pattern of H. trimaculatus protease E hydrolysates fractionated through Hiprep 16/10 DEAE FF ion exchange column chromatograph;

FIG. 6 is a graph illustrating a degree of suppressing a nerve toxin after treating each of five fractions (Fr. I, Fr. II, Fr. III, Fr. VI, Fr. V) fractionated through an ion exchange column chromatograph in nerve toxin-induced PC12 cells;

FIG. 7 is a graph illustrating a sub-fractionated pattern of the fifth fraction (Fr. V) among five factions fractionated through an ion exchange column chromatograph using a high-performance liquid chromatography (HPLC);

FIG. 8 is a graph illustrating a degree of suppressing a nerve toxin after treating each of five sub-fractions (Fr. V-1, -2, -3, -4, -5) fractionated through a high-performance liquid chromatography (HPLC) by using MTT assay in nerve toxin-induced PC12 cells;

FIGS. 9 and 10 are graphs illustrating peaks of single peptide components isolated/purified by using a reverse-phase high-performance liquid chromatography (RP-HPLC) with second and third fractions among five sub-fractions fractionated through HPLC;

FIG. 11 illustrates an amino acid sequence and a molecular weight spectrum of HTP-1, the peptide of the present invention (MS/MS analysis was performed on a Q-TOP tandem mass spectrometer equipped with a nano-ESI source and an amino acid sequencing was done using the PepSeq de novo sequencing algorithm);

FIG. 12 illustrates an amino acid sequence and a molecular weight spectrum of HTP-2, the peptide of the present invention (MS/MS analysis was performed on a Q-TOP tandem mass spectrometer equipped with a nano-ESI source and an amino acid sequencing was done using the PepSeq de novo sequencing algorithm);

FIG. 13 is graphs illustrating BV2 cell viabilities after treating each of the peptides according to the present invention (HTP-1, HTP-2) with various concentrations (10, 50, 100 μM) to microglia, BV2 cells though MTT assay;

FIG. 14 is a graph illustrating BV2 cell viability after treating Aβ42 with various concentrations (0.1, 0.5, 1, 5, 10 μM) to microglia, BV2 cells though MTT assay;

FIG. 15 is a schematic diagram illustrating the co-culture system with BV2 cells and PC12 cells;

FIG. 16 is graphs illustrating PC12 cell viabilities according to treatments of the peptides of the present invention (HTP-1, HTP-2) per concentration at the time of co-culturing BV2 cells and PC12 cells (PC12 cells differentiated from NGF) through MTT assay (Blank: PC12 cells cultured without BV2 cells, Control: PC12 cells co-cultured without the peptide of the present invention);

FIG. 17 is a graph illustrating a degree of alleviating oxidative stress in BV2 cells through DCF fluorescence measurement when stimulating with Aβ42 after pre-treating with HIP-1, the peptide of the present invention with various concentrations (10, 50, 100 μM) to BV2 cells;

FIG. 18 is a graph illustrating a change of NO production amount when stimulating with Aβ42 after pre-treating with HIP-1, the peptide of the present invention with various concentrations (10, 50, 100 μM) to BV2 cells;

FIG. 19 is graphs illustrating changes of inflammation-related mediating factors (PGE2, TNF-α, IL-6, IL-1β) through an enzyme immune analysis when stimulating with Aβ42 after pre-treating with HIP-1, the peptide of the present invention with various concentrations (10, 50, 100 μM) to BV2 cells;

FIG. 20 is RT-PCR images illustrating changes of mRNA expressing amount of inflammation-related mediating factors (iNOS, COX-2, TNF-α, IL-6, IL-1β) through a RT-PCR when stimulating with Aβ42 after pre-treating with HIP-1, the peptide of the present invention with various concentrations (10, 50, 100 μM) to BV2 cells;

FIG. 21 is western blot images illustrating changes of protein expressing amounts of inflammation-related mediating factors (iNOS, COX-2, TNF-α, IL-6, IL-1β) through a western blot analysis when stimulating with Aβ42 after pre-treating with HIP-1, the peptide of the present invention with various concentrations (10, 50, 100 μM) to BV2 cells;

FIG. 22 is graphs illustrating changes of cathepsin B and cathepsin D production amounts through an enzyme immune analysis when stimulating with Aβ42 after pre-treating with HTP-1, the peptide of the present invention with various concentrations (10, 50, 100 μM) to BV2 cells;

FIG. 23 are images illustrating changes of cathepsin B and cathepsin D expression amounts through a RT-PCR (A) and a western blot (B) when stimulating with Aβ42 after pre-treating with HTP-1, the peptide of the present invention with various concentrations (10, 50, 100 μM) to BV2 cells;

FIG. 24 are graphs illustrating changes of anti-inflammatory cytokines, IL-4 and IL-13 production amounts when stimulating with Aβ42 after pre-treating with HTP-1, the peptide of the present invention with various concentrations (10, 50, 100 μM) to BV2 cells; and

FIG. 25 are images illustrating changes of anti-inflammatory cytokines, TGF-β, IL-4, and IL-13 expression amounts through a RT-PCR (A) and a western blot (B) when stimulating with Aβ42 after pre-treating with HTP-1, the peptide of the present invention with various concentrations (10, 50, 100 μM) to BV2 cells.

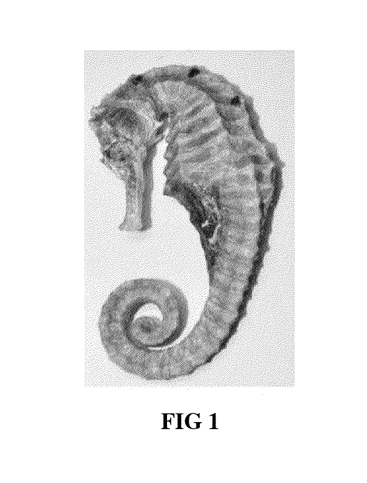

### DETAILED DESCRIPTION OF THE PREFERRED EMBODIMENT

The present invention is characterized for providing a composition for preventing or treating (improving) a neurodegenerative disease, the composition including seahorse protein hydrolysates as an effective component.

The seahorse protein hydrolysates according to the present invention may be obtained through a series of processes for treating the known protease to seahorses.

According to an embodiment of the present invention, such seahorses may be Hippocampus trimaculatus, and the seahorses protein hydrolysates may be obtained by treating such seahorses with typsin, alpha-chymotrypsin, papain, or protease E as a protease.

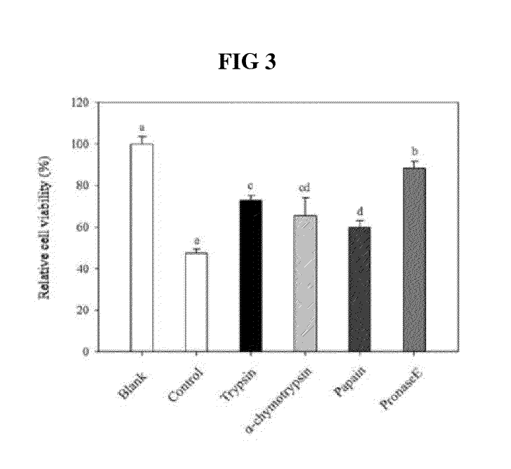

According to the following Example <1-2> of the present invention, the protein hydrolysates were prepared through freeze-drying Hippocampus trimaculatus collected from Indonesia, grinding the dried Hippocampus trimaculatus to prepare it in a type of powder, mixing the Hippocampus trimaculatus powder with each of alpha-chymotrypsin, papain, and protease E in a weight ratio of 1:100 relative to the powder, and reacting for a certain time to be inactivated. As a result, trypsin hydrolysates of Hippocampus trimaculatus, alpha-chymotrypsin hydrolysates of Hippocampus trimaculatus, papain hydrolysates of Hippocampus trimaculatus, and protease E hydrolysates of Hippocampus trimaculatus could be obtained. The experiments were performed for confirming whether or not four hydrolysates described above have all effects of protecting brain cells (see FIG. 3).

In addition, the present invention provides a composition for preventing or treating a neurodegenerative disease, the composition including the peptide having the amino acid sequence set forth in SEQ ID NO: 1 or 2 as an effective component.

The peptide of the present invention can be obtained by performing a series of processes or prepared through a chemical synthetic method that is known in the prior art, from Hippocampus trimaculatus, seahorses.

In a case of obtaining the peptide of the present invention from Hippocampus trimaculatus, the peptide can be prepared by performing as follows: (a) preparing through freeze-drying seahorses, Hippocampus trimaculatus and then grinding it in a type of powder; (b) preparing seahorse protease E hydrolysates through adding protease E to the powder and then reacting; (c) fractionating the seahorses protease E hydrolysates using an ion exchange column chromatograph; (d) further purifying the fractions using a high-performance liquid chromatography (HPLC); and (e) isolating the purified fractions into a single peptide component using a reverse-phase HPLC (RP-HPLC).

The present inventors confirmed that the peptide including the amino acid sequence set forth in SEQ ID NO: 1 or 2 can protect brain cells from a nerve toxin and oxidative stress, suppress expressions of inflammation mediating factors, and increase an expression of anti-inflammatory cytokines, thereby having excellent anti-inflammatory activity, and also excellent effects of suppressing expressions of cathepsin B and cathepsin D involving in a production process of Aβ42, a nerve toxin. Accordingly, the present inventors primordially identified the fact in that the peptide is effective for preventing or treating (improving) a neurodegenerative disease.

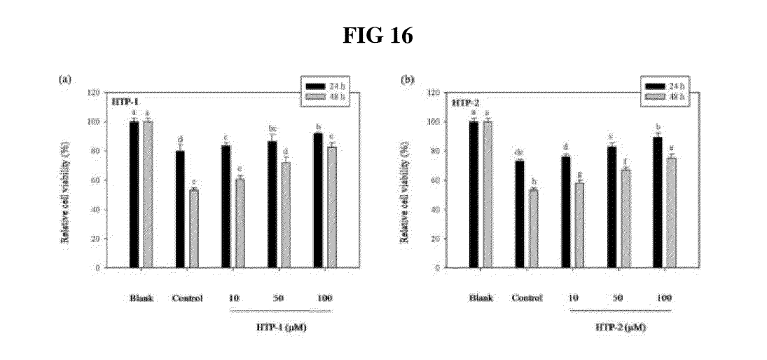

More particularly, according to the following Example <2-3>, whether or not the peptide including the amino acid sequence set forth in SEQ ID NO: 1 or 2 of the present invention can protect brain cells was confirmed. As a result, it could be confirmed that the peptide of the present invention can increase effect of protecting brain cells through confirming cell viability increase of brain cells depending on the treated concentrations (see FIG. 16).

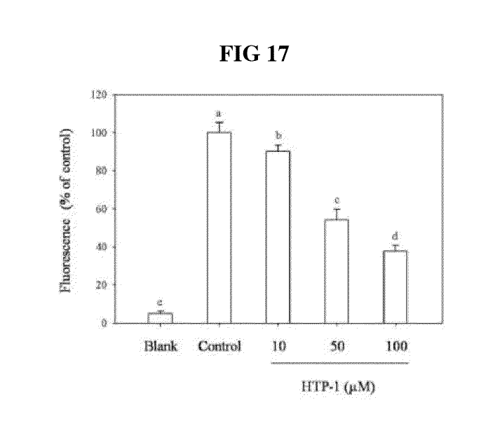

In addition, according to the following Example <2-4>, whether or not the peptide including the amino acid sequence set forth in SEQ ID NO: 1 of the present invention can suppress intracellular oxidative stress was confirmed. As a result, it could be confirmed that the peptide of the present invention can suppress active oxygen species in BV2 cells, murine microglia depending on the treated concentrations (see FIG. 17).

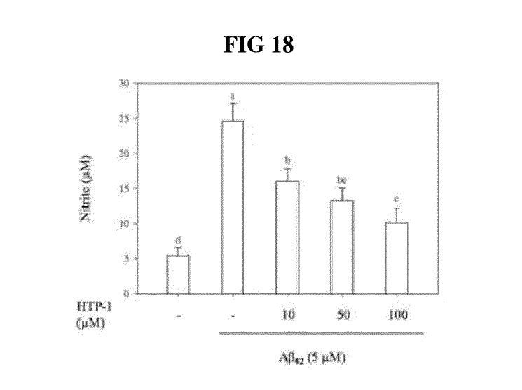

In addition, according to the following Examples <2-5> to <2-7>, whether or not the peptide including the amino acid sequence set forth in SEQ ID NO: 1 of the present invention can suppress inflammation-related mediating factors was confirmed. As a result, it could be confirmed that the peptide of the present invention can decrease amounts of NO, PGE2, iNOS, COX-2, TNF-α, and IL-1β productions and expressions depending on the treated concentrations (see FIGS. 18 to 21).

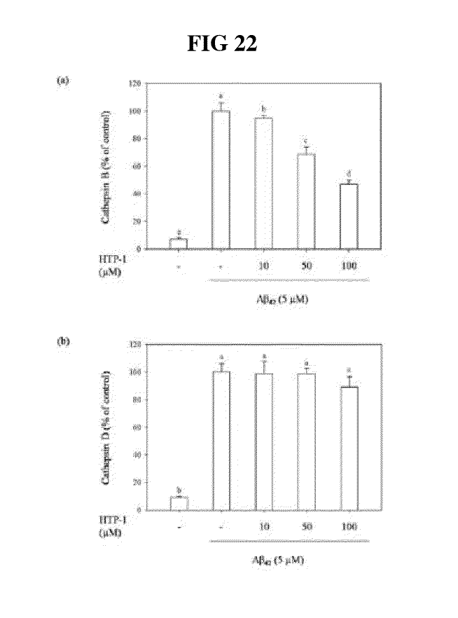

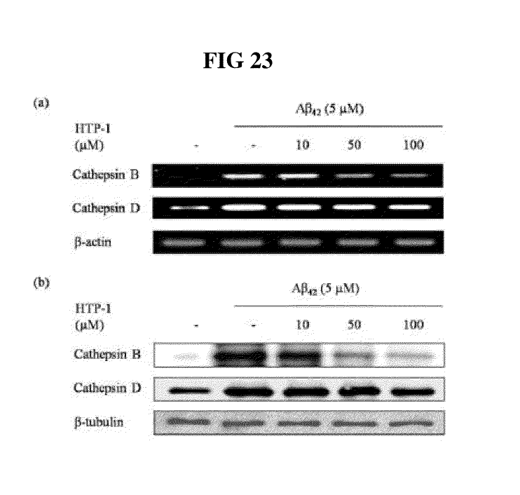

In addition, according to the following Example <2-8>, whether or not the peptide including the amino acid sequence set forth in SEQ ID NO: 1 of the present invention can suppress cathepsin B and cathepsin D, protease (involving in a process of producing Aβ42) was confirmed. As a result, it could be found that the peptide of the present invention can decrease amounts of cathepsin B and cathepsin D productions and expressions depending on the treated concentrations (see FIGS. 22 and 23).

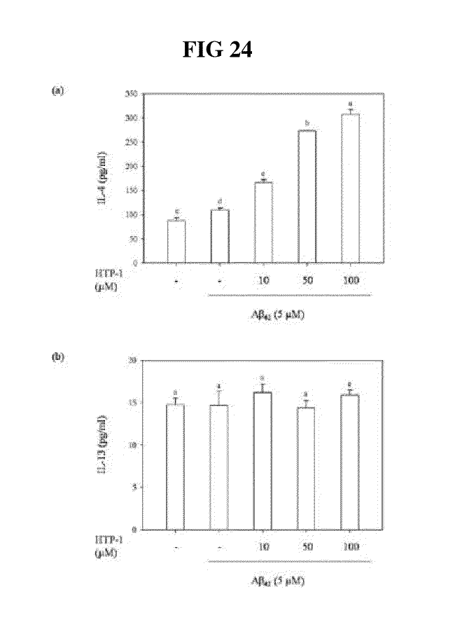

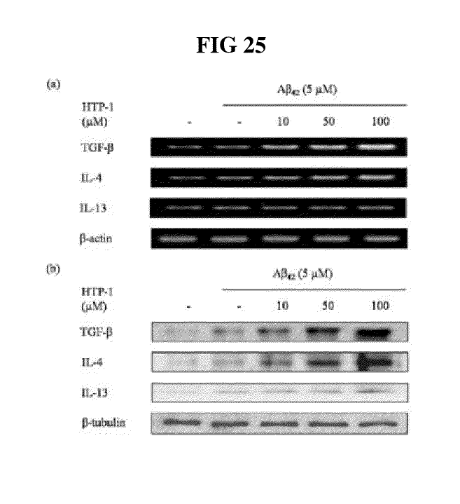

In addition, according to the following Example <2-9>, whether or not the peptide including the amino acid sequence set forth in SEQ ID NO: 1 of the present invention can increase anti-inflammatory cytokines was confirmed. As a result, it could be confirmed that the peptide of the present invention can increase amounts of IL-4 and TGF-β productions and expressions depending on the treated concentrations (see FIGS. 24 and 25).

According to the above-described results, it was experimentally identified that the seahorses protein hydrolysates and peptide having the amino acid sequence set forth in SEQ ID NO: 1 or 2 isolated and purified from the hydrolysates has a protection effect of brain cells. Especially, it was experimentally identified that the peptide having the amino acid sequence set forth in SEQ ID NO: 1 is effective for preventing or treating (improving) a neurodegenerative disease through brain cells protecting activity, oxidative stress alleviating activity, anti-inflammatory activity, and protease suppressing activity.

The composition of the present invention may be a pharmaceutical composition or food composition.

The pharmaceutical composition of the present invention may be prepared by using a pharmaceutically suitable and physiologically acceptable adjuvant in addition to the effective component. The adjuvant may include excipient, a disintegrating agent, a sweeting agent, a binding agent, a coating agent, a swelling agent, a lubricant, a modifier, a flavoring agent, and the like.

The pharmaceutical composition may be preferably formulated into a pharmaceutical composition including further at least one pharmaceutically acceptable carrier in addition to the above-described effective component in order for an administration.

A dosage form of the pharmaceutical composition may include granules, powders, tables, coated tables, capsules, suppository, solutions, syrups, juices, suspensions, emulsions, drops, injections, or the like. For example, in order to formulate in a type of tables or capsules, the effective component may be combined with an oral and non-toxic pharmaceutically acceptable inactive carrier, such as ethanol, glycerol, and water. In addition, if necessary or desired, a suitable binding agent, lubricant, disintegrating agent, and color coupler may be included in a mixture. The suitable binding agent may include natural sugar, such as starch, gelatin, glucose, or beta-lactose, natural and synthetic gum, such as a corn sweeting agent, acacia, tragacanth, or sodium oleate, sodium stearate, magnesium stearate, sodium benzoate, sodium acetate, sodium chloride, and the like, but the present invention is not limited thereto. The disintegrating agent may include starch, methyl cellulose, agar, bentonite, xanthan gum, and the like, but the present invention is not limited thereto. For the composition to be formulated into solution, the pharmaceutically acceptable carrier may include saline solution, distilled water, Ringer's solution, buffer saline solution, albumin injection solution, dextrose solution, malto dextrin solution, glycerol, ethanol, and a mixture in combination with at least one of them, and if necessary, other general additives, such as antioxidant, buffer solution, and bacteristat may be added. In addition, diluents, a distributing agent, surfactant, a binding agent, and a lubricant may be further added to formulate it into injective dosage form, such as aqueous solution, suspensions, and emulsion, pills, capsules, granules, and tables. Furthermore, it may be preferably formulated according to each diseases or components using a method disclosed in Remington's Pharmaceutical Science, Mack Publishing Company, Easton Pa. as a proper method in the prior art.

According to an embodiment of the present invention, the peptide including the amino acid sequence set forth in SEQ ID NO: 1 or 2 of the present invention may be included in a concentration of 10 to 10000 μM in the composition, and 0.1 wt % to 95 wt % relative to the total weight of the composition.

A neurodegenerative disease that may be treated and have a treatment effect by using the pharmaceutical composition of the present invention may include Alzheimer's diseases (AD), Parkinson's diseases (PD), Lou Gehrig's diseases, Huntington's diseases (HD), amyotrophic lateral selerosis (ALS), multiple sclerosis, immune system-damaged brain function abnormalities, progressive nervous diseases, metabolic brain diseases, Niemann-Pick diseases, and dementias caused by ischemic stroke and cerebral hemorrhage, but the present invention is not limited thereto.

The composition of the present invention may be also a food composition. The food composition may include various flavoring agents, natural carbohydrates, or the like as an additive component like the general food composition in addition to the peptide having the amino acid sequence set forth in SEQ ID NO: 1 or 2 as an effective component.

An example of the above-described natural carbohydrates may include general sugar, such as monosaccharide, for example, glucose, fructose, and the like; disaccharide, for example, maltose, sucrose, and the like; and polysaccharide, for example, dextrin, cyclodextrin, and the like; and sugar alcohol such as xylitol, sorbitol, and erythritol. As the above-described flavouring agents, a natural flavouring agent (Thaumatin), Stevia extract (for example, Rebaudioside A, Glycyrrhizine, and the like), and a synthetic flavouring agent (saccharine, aspartame, and the like) may be advantageously used.

The food composition according to the present invention may be used as functional foods or may be added to various foods by formulating using the same method as the pharmaceutical composition. As foods, in which the composition of the present invention can be included, there are for example, drinks, meats, chocolates, foods, confectionery, pizzas, ramen, other noodles, gums, candies, ice creams, alcohol drinks, vitamins, health supplements, and the like.

In addition, such a food composition may include various nutrients, vitamins, minerals (electrolyte), tastes such synthetic tastes and natural tastes, coloring agents and enhancers (cheese, chocolates, and the like), pectic acid and a salt thereof, alginic acid and a salt thereof, organic acid, protective colloid thickeners, pH adjuster, stabilizers, preservatives, glycerin, alcohols, a carbonating agent for a carbonated drink, and the like, in addition to the peptide having the amino acid sequence set forth in SEQ ID NO: 1 or 2, that is an effective component. In addition to that, the food composition of the present invention may include fruit flesh for preparing natural fruit juices, fruit juice drinks, and vegetable drinks.

In addition, the present invention provides a health functional food for preventing or improving a neurodegenerative disease, the food including the peptide having the amino acid sequence set forth in SEQ ID NO: 1 or 2.

The health functional food of the present invention may be prepared and processed in a type of tablets, capsules, powder, granules, liquids, pills, and the like for preventing and improving a neurodegenerative disease.

The term, “health functional foods” disclosed in the present invention relates to foods that are prepared and processed by using a raw material or component having useful functionality to humans under the laws about health functional foods, and means foods that are ingested for controlling nutrients for structures and functions of human body and obtaining a useful effect for health care, such as a physiological effect.

The health functional foods of the present invention may include general food additives, and whether or not the health function foods are suitable is determined based on a standard and criteria relating to a relevant item according to general rules disclosed in Korean Food Additives Codex and a general test method that have been approved by Korea Food & Drug Administration as long as other rules do not provide.

The items disclosed in such “Korean Food Additives Codex” may include, for example, a chemically synthetic composite, such as ketones, glycine, calcium citrate, nicotinic acid, and cinnamic acid; a natural additive material, such as persimmon color, licorice extract, microcrystalline cellulose, Kaoliang color, and guar gum; and mixed formulations, such as sodium L-glutamate formulation, alkali agents for noodles, preservative formulation, and tar color formulation.

For example, the health functional foods in a type of tablets may be prepared by granulating a mixture of the peptide having the amino acid sequence set forth in SEQ ID NO: 1 or 2 that is an effective component of the present invention with excipient, a binding agent, a disintegrating agent, and other additives through a general method, and then compression-molding through adding a modifier, and the like, or directly compression-molding the mixture described above. In addition, the health functional foods in a type of tablets may include flavor enhancers, and the like if necessary.

Among the health functional foods in a type of capsules, a hard capsule of the health functional foods may be prepared by filling the mixture mixing the peptide having the amino acid sequence set forth in SEQ ID NO: 1 or 2 that is an effective component of the present invention with additives such as excipient into a general hard capsule. A soft capsule of the health functional foods may be prepared by filling the mixture mixing the peptide having the amino acid sequence set forth in SEQ ID NO: 1 or 2 with additives such as excipient into a capsule basic material such as a gelatin. The soft capsule formulation may include a plasticizer such as glycerin or sorbitol, a coloring agent, preservatives, and the like if necessary.

The health functional foods in a type of a pill may be prepared by molding the mixture mixing the peptide having the amino acid sequence set forth in SEQ ID NO: 1 or 2 that is an effective component of the present invention with excipient, a binding agent, and a disintegrating agent by using a known method in the prior art, and if necessary, may be coated with white sugar or other coating agents. In addition, the health functional foods in a type of a pill may be coated with a material such as starch or talc.

The health functional foods in a type of a granule may be prepared by preparing the mixture mixing the peptide having the amino acid sequence set forth in SEQ ID NO: 1 or 2 that is an effective component of the present invention with excipient, a binding agent, and a disintegrating agent in a type of a granule by using a known method in the prior art, and if necessary, may include fragrance ingredients, flavor enhancers, and the like.

The health functional foods may be drinks, meats, chocolates, foods, confectionery, pizzas, ramen, other noodles, gums, candies, ice creams, alcohol drinks, vitamins, health supplements, and the like.

The neurodegenerative disease that may exhibit an effect of preventing or improving may include Alzheimer's diseases (AD), Parkinson's diseases (PD), Lou Gehrig's diseases, Huntington's diseases (HD), amyotrophic lateral selerosis (ALS), multiple sclerosis, immune system-damaged brain function abnormalities, progressive nervous diseases, metabolic brain diseases, Niemann-Pick diseases, and dementias caused by ischemic stroke and cerebral hemorrhage, but the present invention is not limited thereto.

In addition, the present invention provides a use of the composition including the peptide having the amino acid sequence set forth in SEQ ID NO: 1 or 2 as an effective component for preparing a medicine or food for preventing or treating (improving) a neurodegenerative disease. The composition including the peptide having the amino acid sequence set forth in SEQ ID NO: 1 or 2 as an effective component according to the present invention may be used for preparing a medicine or food for preventing and treating (improving) a neurodegenerative disease.

In addition, the present invention provides a method for preventing or treating a neurodegenerative disease, the method including administrating the peptide having the amino acid sequence set forth in SEQ ID NO: 1 or 2 to a mammal.

The term “mammal” used in the present specification refers to a mammal that is an object to be treated, observed, or experimented, and preferably human.

The term “therapeutically effective amount” used in the present specification means an amount of an effective component or pharmaceutical composition inducing a biological or medical reaction to a tissue system, animal, or human to be considered by researchers, veterinarians, doctors, or other clinical instructions, and refers to an amount inducing an alleviation of symptoms of diseases or disorders to be treated. The therapeutically effective dosage or dosage number of the effective component according to the present invention may be obvious by the skilled person in the prior art. Accordingly, the optimum dosage to be administrated may be easily determined by the skilled person in the prior art, and may be controlled by various factors such as a type of a disease, severity of a disease, the contents of an effective component and other components included in the composition, a type of dosage form, age of a patient, a body weight of a patient, a general health condition, sex, diets, an administration time, an administration route, a rate of secreting the composition, a treatment period, and drugs to be co-administrated.

According to the treatment method of the present invention, the composition including the peptide having the amino acid sequence set forth in SEQ ID NO: 1 or 2 as an effective component may be administrated in a general route, such as an oral administration, a rectum administration, an intravenous injection, an intra-abdominal injection, a topical injection, an intradermal injection, and an inhale route.

Exemplary embodiments of the present invention will now be described in detail with reference to the accompanying drawings. However, the range of the present invention is not limited to Examples.

### Example

### Reagents

Cell culture medium [Dulbecco's Modified Eagle's Medium (DMEM)], penicillin/streptomycin, fetal bovine serum (FBS), and the other materials required for culturing cells were purchased from Gibco BRL, Life Technologies (Grand Island, US). Horse serum, NGF, bovine serum albumin (BSA), proteolytic enzyme (trypsin, α-chymotrypsin, papain, and protease E), 1,1,1,3,3,3-Hexafluoro-2-Propanol (HFIP), poly-D-lysine, Aβ42, 3-(4,5-dimethylthiazol-2-yl)-2,5-diphenyltetrazolium bromide (MTT), dimethylsulphoxide (DMSO), H₂O₂ were obtained from Sigma-Aldrich (St. Louis, Mo., USA). Other chemical and reagents used in this study were of analytical grade.

Cell Culture

Murine microglia, BV2 cell line was a kind gift of Prof. Il Whan Choi (Inje University, Korea). Rat pheochromocytoma, PC12 cell line was obtained from American Type Culture Collection (Manassas, USA). BV2 cells were grown in DMEM supplemented with 5% FBS, 2 mM glutamine, and 100 μg/ml penicillin-streptomycin; meanwhile PC12 cells were grown in DMEM supplemented with 10% horse serum and 5% FBS, 2 μM glutamine, and 100 μg/ml penicillin-streptomycin.

### Example 1

### Identification of Bioactive Peptide from Hippocampus trimaculatus

<1-1> Seahorses Sample Preparation

H. trimaculatus were collected along the Karimun Jawa Island coast of Indonesia during the period of May 2011. H. trimaculatus collected was identified by Prof. Ocky Karna Radjasa, Ph.D (Marine biologist, Diponegoro University-Indonesia). The samples collected were washed with tap water to remove salt, and dried in the shade for 2 weeks. Dried H. trimaculatus were freeze dried for 3 days and ground until become a powder to be used for the experiment.

<1-2> H. trimaculatus Hydrolysates

The present inventors prepared hydrolysates from the prepared seahorses samples in Example <1-1> using various proteases and then investigated their effect on protecting nerve through a test for suppressing a nerve toxin in order to select hydrolysates having excellent effect on protecting nerve from the seahorses hydrolysates.

In order to get the optimum enzyme to hydrolyze H. trimaculatus, four protease enzyme (trypsin, α-chymotrypsin, papain, and protease E) were used. In brief, cleaned H. trimaculatus (250 mg) prepared in Example <1-1> was mixed with each of trypsin, α-chymotrypsin, papain, and protease E in a weight ratio of 1:100 relative to the H. trimaculatus sample. The mixture was incubated for 6 h with stirring and then heated in a boiling water bath for 10 min to inactivate the enzyme.

A nerve protection effect of each of H. trimaculatus hydrolysates prepared through the above-described process was evaluated by confirming a degree of suppressing a nerve toxin in PC12 cells induced by amyloid-beta 42 (Aβ42). The PC12 cell is a cloned of rat pheochromocytoma cells line that retains a number of chromaffin cell characteristics such as the presence of nicotinic cholinergic receptors, the synthesis and secretion of catecholamines, and the expression of a number of neuropeptide genes. The PC12 cell line is a useful model for the study of neuronal development, since the PC12 cells can be induced to differentiate toward cholinergic neurons after exposure to nerve growth factor (NGF). Recent evidence suggests that Aβ42 induces neuronal apoptosis in the brain and in primary neuronal cultures, and that this Aβ42-induced neuronal death may be responsible in part for the cognitive decline found in AD patients. For this reason, stimulation of PC12 cells with Aβ42 constitutes an excellent model for the screening and subsequent evaluation of the effects of candidate drugs on the neurodegenerative diseases. Hereinafter, the amyloid-beta 42 will be designated by “Aβ42”, simplify.

The present inventors induced a nerve toxin in PC12 cells by differentiating PC12 cells into nerve cells through treating with NGF, and then treating to the differentiated PC12 cells with 20 μM of Aβ42 for 24 hours. Each of H. trimaculatus hydrolysates (trypsin, α-chymotrypsin, papain, and protease E) was treated to the nerve-toxin-induced PC12 cells, and then the degree of suppressing the nerve toxin was measured by using a MTT assay.

Cytoprotective effect was determined by MTT reduction assay. In brief, cells were seeded into 96-well plates at a density of 2×10⁴ cells/well and incubated with serum free medium in the presence of each of H. trimaculatus hydrolysates (trypsin, α-chymotrypsin, papain, and protease E). After incubation for 24 hours, 100 μl of MTT (1 mg/ml final concentration) was added to each of wells and incubation was continued for another 4 hours. MTT is used as an indicator of cell viability through its mitochondrial reduction to formazan. Mitochondrial succinate dehydrogenase in live cells converts MTT into visible formazan crystals during incubation. The formazan crystals were then solubilized in DMSO and the absorbance was measured at 540 nm by using enzyme-linked immunosorbent assay (ELISA) microplate reader (Tecan GmbH, Austria). Relative cell viability was calculated compared with the absorbance of the untreated control group.

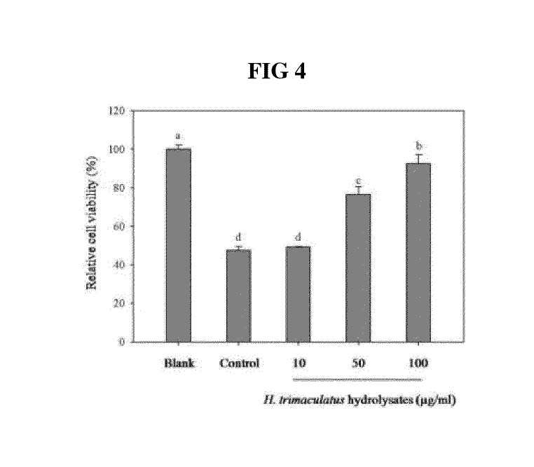

As illustrated in FIG. 3, exposure to Aβ42 at a concentration of 20 μM for 24 h resulted reduction in PC12 cell viability. Interestingly, all four hydrolysates showed cytoprotective effect to PC12 from Aβ42 neurotoxic effects; with protease E hydrolysate exerted highest cytoprotective effect (88.33±3.33%). In addition, as illustrated in FIG. 4. PC12 cells were treated with different concentrations of the H. trimaculatus hydrolysates. The cell viability data confirmed that H. trimaculatus hydrolysates have cytoprotective effect on Aβ42-induced cell death depending on the concentrations.

<1-3> Reaction Optimization for Enzyme Hydrolysis

The present inventors performed a reaction surface analysis in order to confirm the optimum conditions of the reaction for seahorse protein hydrolysis, and obtained the optimum conditions for hydrolysis. The results are shown in the following Table 1.

TABLE 1
Optimal Condition of Enzyme Reaction for
H. Trimaculatus Protein Hydrolysis

| | target | | predicted |
|---|---|---|---|
| responses | value | optimal conditions | value |
| cell | maximum | X1 = 36.69 (Tem.) | 94.61 ± 2.12% |
| viability (Y) | | X2 = 20.01 h (Hour) | |
| | | X3 = 2.02% (Enzyme) | |
| | | X4 = 7.34 (pH) | |

<1-4> Isolation of Peptide Having Activity from H. trimaculatus Protease E Hydrolysates

It was confirmed that the hydrolysates hydrolyzed by using protease E among enzymes as enzyme in the H. trimaculatus hydrolysates obtained from Example <1-2> was most effective for protecting the nerve toxin-induced PC12 cells. Accordingly, in order to obtain bioactive peptide from such H. trimaculatus protease E hydrolysates, the present inventors isolated the final peptide of the present invention from the fraction exhibiting excellent cytoprotective effect among all the fractions fractionated through a phased chromatography.

First, 40 mg/ml of the dried H. trimaculatus protease E hydrolysates was dissolved in 20 μM sodium acetate (pH 4) and loaded into a Hiprep 16/10 DEAE FF anion exchange column using a linear gradient of NaCl (0-2.0M). Elution peak were monitored at 280 nm and each fractions was collected every 5 ml. Since then, all fractions according to peaks were divided and then concentrated by using a rotary vacuum evaporator.

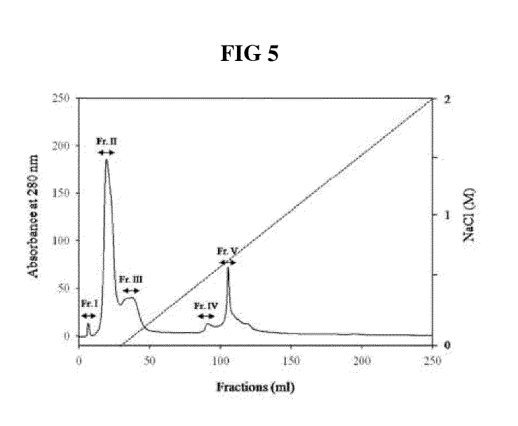

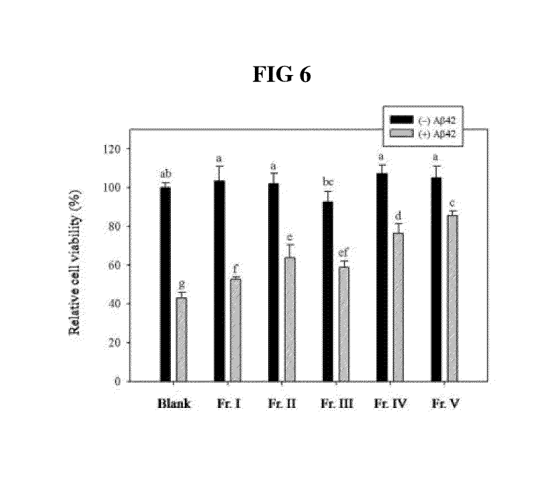

As illustrated in FIG. 5, the H. trimaculatus protease E hydrolysates spectrum was separated into five fractions. Cytoprotective activities of all fractions were measured by confirming degrees of suppressing nerve toxin through treating each of the fractions to the nerve toxin-induced PC12 cells. As a result and illustrated in FIG. 6, among five fraction tested, fraction V (Fr. V) possess the highest protection against Aβ42-induced cell death with the cell viability value of 85.52±2.22%.

For this reason, Fr. V also exhibiting highest protection effect was further purified, and gradient of acetonitrile (0-30%) was chosen. At this time, an eluent rate was 1.0 ml/min. The eluent peak was detected at 215 nm, and active peak was concentrated by using a rotary evaporator.

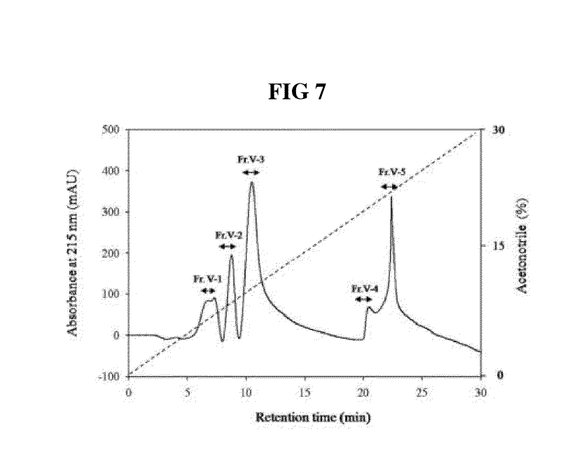

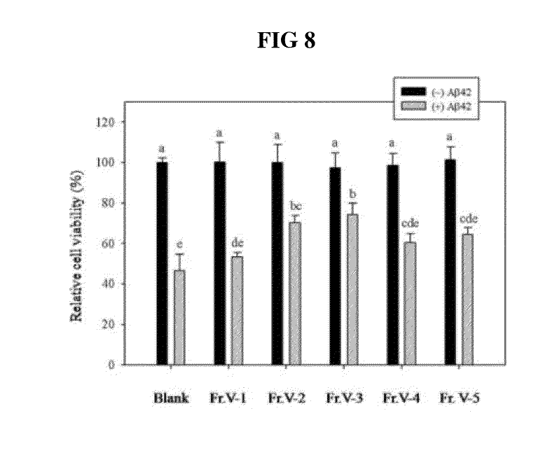

As illustrated in FIG. 7 as a result, five sub-factions (Fr V-1, Fr V-2, Fr V-3, Fr V-4, and Fr V-5) were isolated and treated to the nerve toxin-induced PC12 cells to measure cytoprotective effects as a degree of suppressing a nerve toxin. As a result, in FIG. 8, PC12 cells treated with Fr. V-2 and Fr. V-3 showed highest protective effect against Aβ42 induced neurotoxicity with the value of 79.43±3.59% and 74.23±5.67%, respectively.

Finally, the present inventors purified a single peptide component from Fr V-2 and Fr V-3 fractions through the above-described process using a reverse phase high performance liquid chromatography (RP-HPLC) (Dionex Ltd., Sunnyvale, Calif., USA). Specifically, the fraction exhibiting the strongest radical scavenging activity was further purified using reversed-phase high performance liquid chromatography (RP-HPLC) (Dionex Ltd., Sunnyvale, Calif., USA) on a Nucleosil C18 (10 mm×250 mm, Mackerey Nagel-Nucleosil, Duren, Germany) column with a linear gradient of acetonitrile (0-20% in 45 min) at a flow rate of 1.0 ml/min. Elution peaks were detected at 215 nm, and the active 26 peak was concentrated using a rotary evaporator.

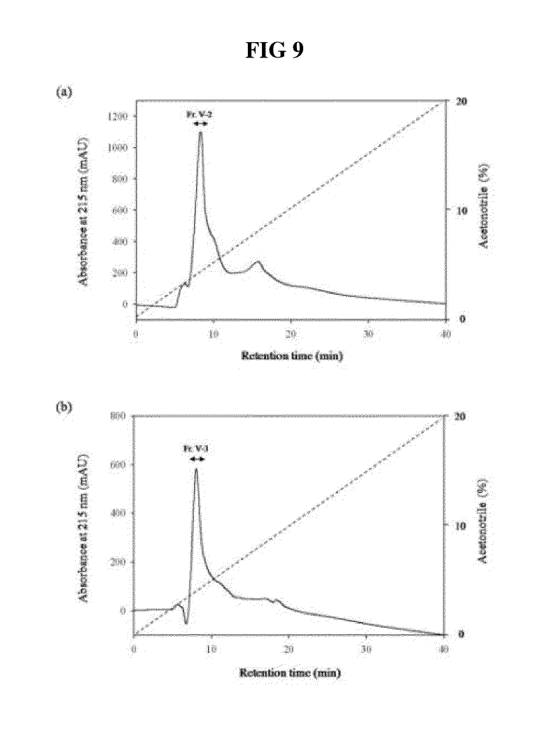

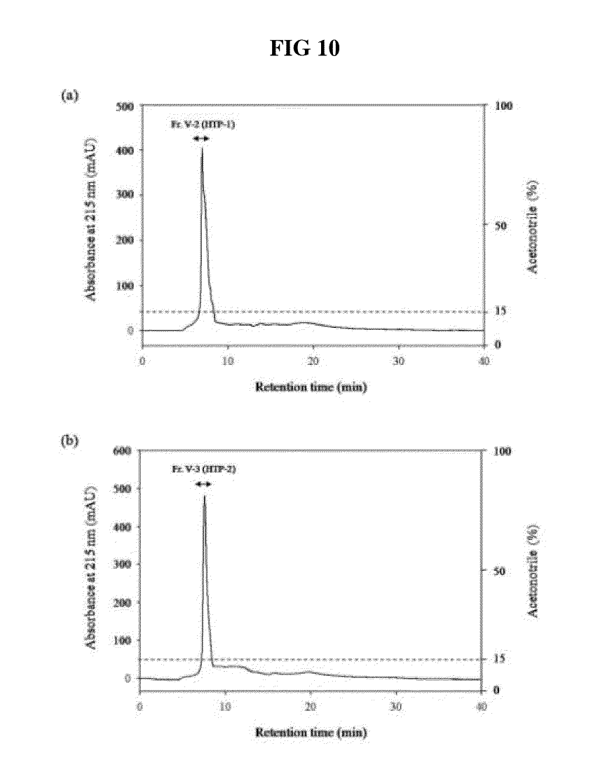

As a result, as illustrated in FIGS. 9 and 10, a single peptide component could be purified from Fr. V-2 and Fr. V-3 fractions.

<1-5> Identification of Bioactive Peptide

Amino acid sequencing was performed with the peptide obtained and purified from Example <1-4>.

The accurate molecular mass and amino acid sequence of the purified peptide was determined by Peptron, Inc. (Daejeon, Korea) using the Q-TOF mass spectrometer (Micromass, Altrincham, UK) coupled with an electrospray ionization (ESI) source. The purified peptides in Example <1-4> were separately infused into the electrospray source after being dissolved in methanol/water (1:1, v/v), and its molecular mass was determined by the doubly charged (M+2H)2+ state in the mass spectrum.

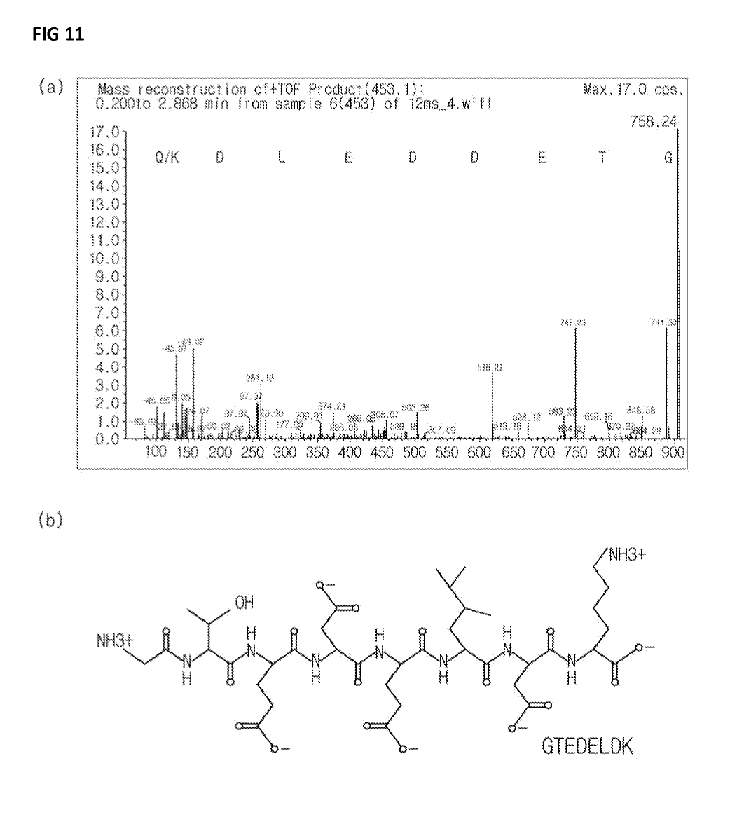

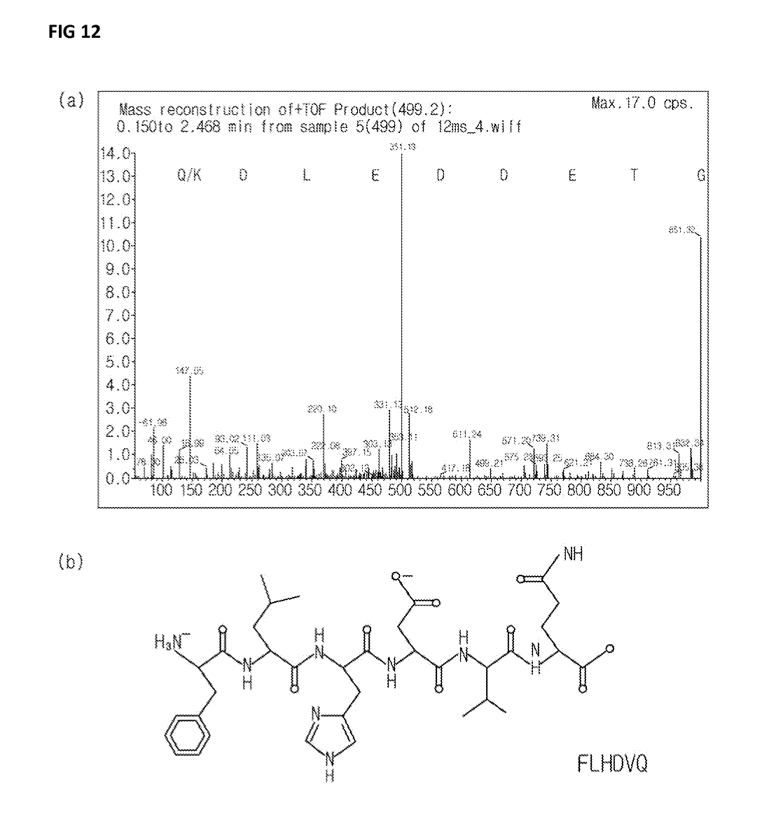

As a result, as illustrated in FIGS. 11 and 12, peptide isolated from H. trimaculatus was identified as HTP-1 (GTEDELDK) and HTP-2 (FLHDVQ). The HTP-1 composed of 8 amino acids with 906.4 Da molecular weight and +25.39 Kcal/mol hydrophobicity. Meanwhile HTP-2 composed of 6 amino acids with 997.4 Da molecular weight and +11.22 Kcal/mol hydrophobicity.

TABLE 2
Peptide of present invention

| Peptide of Present | | |
|---|---|---|
| Invention | Amino Acid Sequence | SEQ ID NO: |
| HTP-1 | GTEDELDK | SEQ ID NO: 1 |
| HTP-2 | FLHDVQ | SEQ ID NO: 2 |

### Example 2

### Bioactivity Test of Peptide of Present Invention on Brain Disease Model

In order to test an effect of the peptide according to the present invention, in which the peptide was identified through Example 1, the present inventors investigated cell toxicity evaluation, cytoprotective effect, effect of suppressing active oxygen species, and anti-inflammatory effect after treating with the peptide of the present invention to the brain disease model.

<2-1> Evaluations of Cytotoxicity of HTP-1 and HTP-2 of Present Invention

For the test, cell viability of BV2, murine microglia, was measured through MTT assay in order to confirm whether or not the peptide HTP-1 and HTP-2 of the present invention isolated and purified through Example 1 were a safe material, so that they did not exhibit toxicity in brain cells.

Cytoprotective effect was determined by MTT reduction assay. In brief, first, BV2 cells, murine microglia, were seeded into 96-well plates at a density of 2×10⁴ cells/well and incubated with serum free medium in the presence of the peptides HTP-1 and HIP-2 of the present invention with different sample concentrations (10, 50, 100 μM). After incubations for 24 hours and 48 hours, the incubated cells were washed with PBS, 100 μl of MTT (0.5 mg/ml final concentration) was added to each wells, and incubation was continued for another 4 hours for a reduction of MTT. MTT is used as an indicator of cell viability through its mitochondrial reduction to formazan. Mitochondrial succinate dehydrogenase in live cells converts MTT into visible formazan crystals during incubation. The formazan crystals were then solubilized in DMSO and the absorbance was measured at 540 nm by using enzyme-linked immunosorbent assay (ELISA) microplate reader (Tecan GmbH, Austria). Relative cell viability was calculated as a percentage compared with the absorbance of the untreated control group.

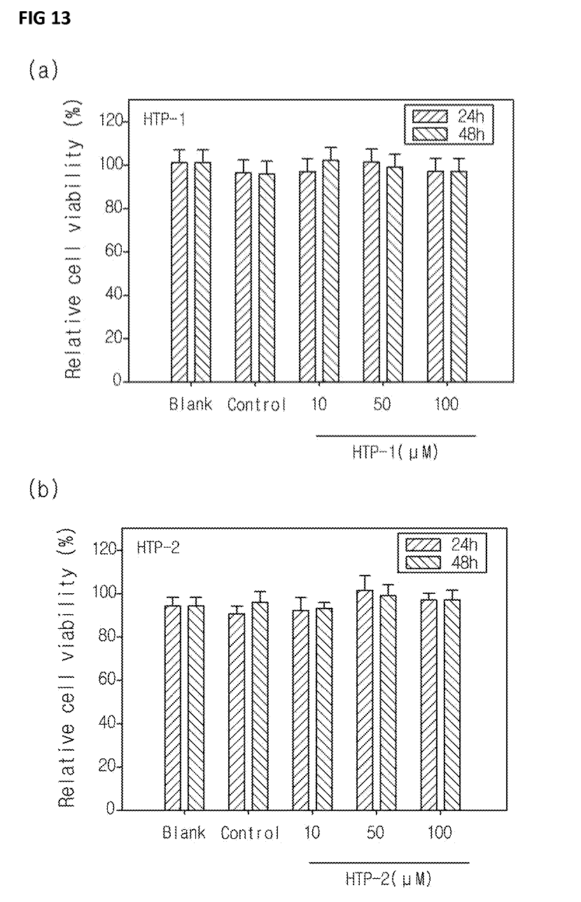

As a result, as illustrated in FIG. 13, it could be confirmed that the peptides of the present invention, HTP-1 and HTP-2 did not exhibit cytotoxicity in BV2 cell at all the treated concentrations. Accordingly, it could be confirmed from the above-described results that the peptides of the present invention, HTP-1 and HTP-2 were safe at a concentration of 10 to 100 μM.

<2-2> Effect of Aβ42 on BV2 Cells

For the experiment, in order to confirm effect of Aβ42 that is well-known as a material exhibiting a nerve toxin, and good cohesiveness, and also inducing apoptosis of brain cells on BV2 cells, murine microglia, Aβ42s with different concentrations were treated to BV2 cells and then the cell viability was measured through MTT assay.

MTT assay was performed by using the same method as Example <2-1>, but the peptides HTP-1 and HTP-2 were used as a sample instead of Aβ42.

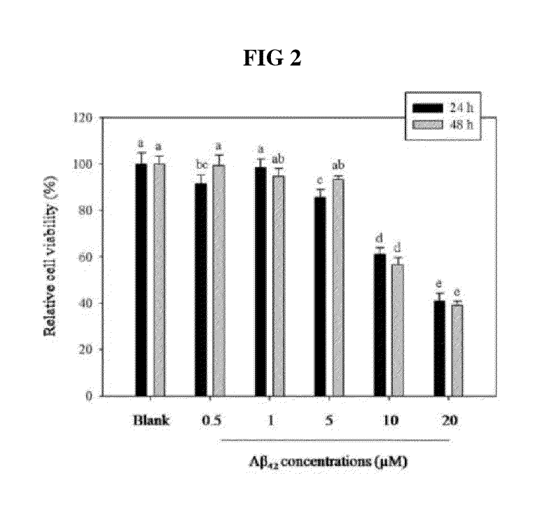

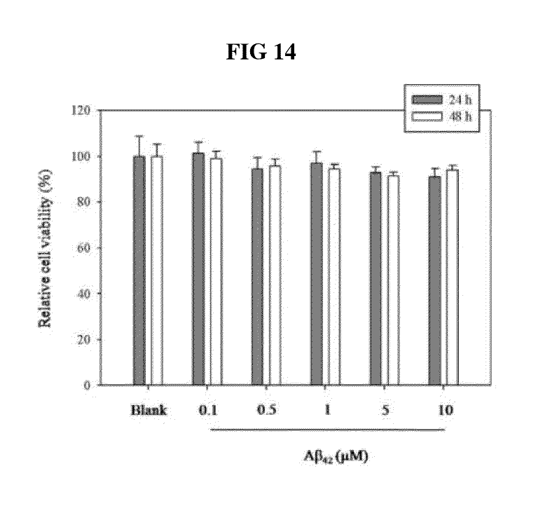

As a result, as illustrated in FIG. 14, it could be confirmed that there were no toxicities in BV2 cells at all the treated concentrations of Aβ42. Such a result is contrast to the result that is a greatly decreased cell viability of the nerve cell-differentiated PC12 cells according to the treatment of 10 μM concentration of Aβ42 (see FIG. 2).

<2-3> Effect of HTP-1 and HTP-2 Peptides of Present Invention on Nerve Toxicity of Activated BV2

In general, other kinds of cells such as glial cells are present at an entangled neuron or folded neuron. The majority of glial cell is distributed in brain, and the size of glial cell is about 1/10 of neuron, but the number of glial cells is about 10 times as compared with the number of neurons. The glial cells play important roles in neuron, and there are 10 glial cells per one neuron. The glial cell is in charge of binding a neuron tissue, supporting a neuron tissue, and supplying nutrition to a neuron tissue. A type of the glial cell is oligodentroglia, astrocyte, and microglia, and especially, it is known that microglia is neuroblast involving in an immune function in brain.

For the present invention, in order to make the condition in vitro to be similar as the condition in vivo, PC12 cells that have been differentiated and are a kind of neuron cells and BV2 cells that is microglia were co-cultured, and then Aβ42 and HTP-1 and HTP-2 peptides of the present invention were treated to activate. For the above-activated cells, neurotoxicity was evaluated and effect of the peptides of the present invention on suppressing neurotoxicity was measured.

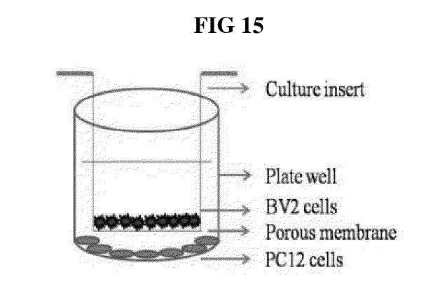

For the co-culture of microglia and PC12 cells, which has been employed to assess the neurotoxin effect of activated microglia, microglia was first seeded onto cell culture inserts (pore size of 0.2 μm; SPL, Korea). After overnight incubation, microglia were left untreated or exposed 5 uM Aβ42 in the presence or absence of peptides for 24 h. Afterwards, the microglial cells were co-cultured with PC12 cells by transferring the culture inserts containing microglia onto PC12 cell monolayers (see FIG. 15).

In order to confirm cell viability of co-cultured PC12 cell, MTT assay was performed. MTT assay was performed by using the same method as that of Example <2-1>.

As a result, as illustrated in FIG. 16, it was confirmed that cell viability in the PC12 cells (PC12 cells that were differentiated with NGF) co-cultured with BV2 cells was decreased as time passed (Control). In addition, it was confirmed that cell viability was increased depending on the treated concentration of HTP-1 and HIP-2 peptides of the present invention. Especially, in the case of treating with 100 μM of each of HTP-1 and HTP-2 peptides for 48 hours, the cell viabilities were 82.49±3.05% and 75.19±2.55%, respectively.

From the above-described results, it could be predicted that in the case of BV2 cells stimulated with Aβ42, the survival of PC12 cells differentiated into neuron cells may be influenced by secreting the factors capable of damaging neuron, and the HTP-1 and HTP-2 peptides of the present invention may decrease such neurotoxicity. Accordingly, the present inventors investigated effect of HTP-1 peptide of the present invention on BV2 cells stimulated (activated) by Aβ42 to be described below in order to verify such assumption through the experiment.

<2-4> Effect of HTP-1 on Intracellular Oxidative Stress Suppression According to a Pre-Treatment of HTP-1 Peptide of Present Invention for Activating BV2 Cells by Stimulating Aβ42

For a process of BV2 cells activation by stimulating Aβ42 in the experiment, free radical scavenger activity was measured in order to confirm effect of HTP-1 peptide on the intracellular oxidative stress suppression according to a pre-treatment of HTP-1 peptide of the present invention. A level of producing an intracellular reactive oxygen species (ROS) in BV2 cells was detected by using 2′,7′-dichlorofluorescein diacetate (DCFH-DA) that is an oxidation-sensitive dye.

The substrate was 2′,7′-dichlorofluorescein diacetate (DCFH-DA) which easily diffuses into the cells and deacetylated by cellular esterase to the more hydrophyllic, nonfluorescent DCFH. The hydrolyzed product was remained in a cell because it has charge thereby not releasing outside a cell. DCFH, a substrate is not fluorescent material, but the oxidative product has strong fluorescence thereby easily being detected. DCFH is oxidized into DCF because of various free radical functions, in which the free radical may be produced under oxidative stress in a cell, such as superoxide anion, H₂O₂, peroxy radical, peroxinitrite, nitric oxide, and the like. Therefore, using the above-described thing, it is possible to quantify total oxidative stress in a cell by measuring DCF fluorescence degree.

First, BV2 cells were seeded into 96-well plates and then cultured for 24 hours, the HTP-1 peptide of the present invention was pre-treated with different sample concentrations (10, 50, 100 μM). After incubations for 6 hours, they were exposed to 5 μM concentration of Aβ42. Since then, the incubated cells were treated with 20 μM DCFH-DA dissolved in PBS, and then reacted under 37° C. and 5% CO₂ humidity for 30 minutes in a dark room. Finally, the cells were washed with PBS twice, and then the fluorescence of DCF dissolved in PBS was measured using GENios fluorescence microplate reader (Tecan Austria GmbH, Salzburg, Austria) at 485 nm excitation and 535 nm emission. Blank was not treated, and control was activated BV2 cells by exposing Aβ42 without the HTP-1 peptide of the present invention.

As a result, as illustrated in FIG. 17, it was confirmed that in the experimental group treated with the HTP-1 peptide of the present invention as compared with the control group, the reactive oxygen species were suppressed depending on the concentration. From the result, it could be confirmed that the HTP-1 peptide of the present invention can alleviate the oxidative stress in a cell.

<2-5> Effect of HTP-1 Peptide on Suppressing Inflammation-Related Mediating Factors According to a Pre-Treatment of HTP-1 Peptide of Present Invention for Activating BV2 Cells by Stimulating Aβ42

For a process of activating BV2 cells by stimulating Aβ42 in the experiment, we confirmed that whether or not the HTP-1 peptide of the present invention affected to amounts of the inflammation-related mediating factors, such as NO, PGE2, TNF-α, IL-6, and IL-1β productions.

NO levels in the cultured BV2 cells' supernatants were measured by the Griess reaction. In brief, BV2 cells were pre incubated overnight in 24-well plates using DMEM without phenol red at a density 2×10⁵ cells/ml, followed by the treatment with the HIP-1 peptide of the present invention having various concentrations (10, 50, 100 μM) as a sample for 6 h. After 6 h, the NO production was stimulated by adding Aβ42 (1 μM final concentration) and incubated for further 48 h. Then 50 μl of culture supernatants from each samples was mixed with the same volume of the Griess reagent [1% sulfanilamide/0.1% N-1-naphthyl]-ethylenediamine dihydrochloride/2.5% phosphoric acid] following the incubation for 15 min. Absorbance values were read at 540 nm using ELISA microplate reader (Tecan GmbH, Austria). Blank was not treated, and control was activated BV2 cells by exposing Aβ42 without the HIP-1 peptide of the present invention.

Assessment of PGE2 synthesis was performed by enzyme immunoassay without prior extraction or purification using commercially available PGE2 enzyme immunometric assay kit (R&D Systems Inc, Minneapolis, USA). BV2 cells were treated with the HIP-1 peptide of the present invention for 6 h and stimulated with Aβ42 (1 μM) for 24 h. The conditioned media was collected to perform PGE2 enzyme immuno-metric assay (PGE2-EIA) according to the instructions of the manufacturer. The concentration of PGE2 was calculated according to the equation obtained from the standard curve plot using PGE2 standard solution in the EIA kit.

Furthermore, the effects of bioactive peptides on the production of pro-inflammatory cytokines were determined by using a pro-inflammatory cytokines analysis kit. BV2 cells were treated with the HIP-1 peptide of the present invention for 6 h and stimulated with Aβ42 (1 μM). The cell supernatants were collected and analyzed as per the manufacturer's instruction with Quantikine mouse TNF-α, IL-6 and IL-1β Immunoassay (R&D Systems Inc, Minneapolis, USA).

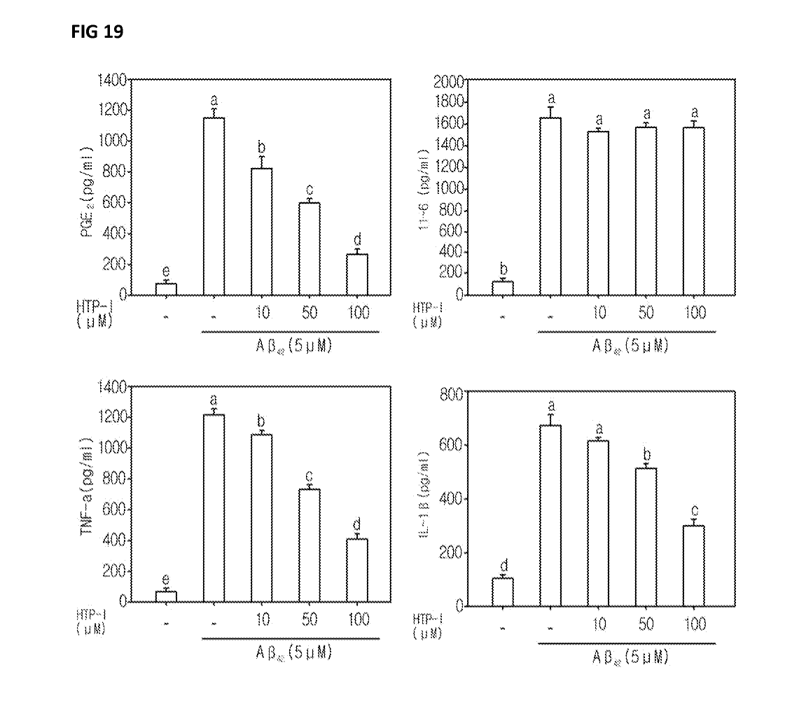

As a result, as illustrated in FIG. 18, the HIP-1 peptide of the present invention was shown to be able to inhibit NO production depending on the treatment concentration. That is, stimulation with Aβ42 markedly induced the production of NO (24.59±2.54 μM) compared to non-stimulated one (5.45±1.14 μM). The attenuation of NO releases varied dependently on HIP-1 concentration. NO levels upon treatment with 10, 50 and 100 μM of HIP-1 were 16.05±1.75 μM, 13.27±1.81 μM, and 10.17±2.06 μM, respectively. It is considered that the decrease of NO production according to the treatment of the HIP-1 peptide is because of the decrease of iNOS expression. Accordingly, it was confirmed as the following tests. In addition, as illustrated in FIG. 19, it was also confirmed that the amounts of PGE2, TNF-α, and IL-1β productions were decreased depending on the treated concentration of HIP-1 peptide of the present invention.

<2-6> Effect of HIP-1 Peptide on Expressing Inflammation-Related Mediating Factors mRNA According to a Pre-Treatment of HIP-1 Peptide of Present Invention for Activating BV2 Cells by Stimulating Aβ42

For a process of activating BV2 cells by stimulating Aβ42 in the experiment, effects of the HTP-1 peptide of the present invention on iNOS, COX-2, TNF-α, IL-6, and IL-1β mRNA expressions that are inflammation-related mediating factors were confirmed by using RT-PCR.

First, the HTP-1 peptides of the present invention with different concentrations were treated to BV2 cells. After 2 hours, Aβ42 (5) was treated and then cultured for 24 hours. The total cell RNA cultured described above was isolated by using Trizol reagent (Invitrogen Co., CA, USA). Equal amount of RNA (2 μg) was used for each cDNA synthesis reaction. It was reverse-transcripted by using 1 μl oligo dT primer (Promega, Madison, Wis., USA). A target cDNA was amplified by using forward primers and reverse primers listed in the following Table 3. Since then, 30 cycles of 95° C. for 45 seconds, 60° C. for 50 seconds, and 72° C. for 5 minutes was performed for amplification. After amplifying, PCR products were then electrophoresed on 1.5% agarose gels for 10 minutes at 100V, and visualized by 1 mg/ml of ethidium bromide staining and quantified using AlphaEAse® gel image-analysis software (Alpha Innotech, San Lendro, Calif., USA). β-actin was used as a control group.

TABLE 3
Primer sequence-1 for RT-PCR

| Gene | Primer sequence | |
|---|---|---|
| iNOS | Forward | 5′-CCC-TTC-CGA-AGT-TTC-TGG-CAG- |
| | | CAG-C-3′ |
| | | (SEQ ID NO: 3) |
| | Reverse | 5′-GGC-TGT-CAG-GCC-TCG-TGG-CTT- |
| | | TGG-3′ |
| | | (SEQ ID NO: 4) |
| COX-2 | Forward | 5′-GGG-GTA-CCT-TCC-AGC-TGT-CAA- |
| | | AAT-CTC-3′ |
| | | (SEQ ID NO: 5) |
| | Reverse | 5′-GAA-GAT-CTC-GCC-AGG-TAC-TCA- |
| | | CCT-G-3′ |
| | | (SEQ ID NO: 6) |
| TGF-α | Forward | 5′-ATG-AGC-ACA-GAAAGC-ATG-ATC-3′ |
| | | (SEQ ID NO: 7) |
| | Reverse | 5′-TAC-AGG-CTT-GTC-ACT-CGA-ATT-3′ |
| | | (SEQ ID NO: 8) |
| IL-1β | Forward | 5′-ATG-GCA-ACT-GTT-CCT-GAA-CTC- |
| | | AAC-T-3′ |
| | | (SEQ ID NO: 9) |
| | Reverse | 5′-TTT-CCT-TTC-TTA-GAT-ATG-GAC- |
| | | AGG-AC-3′ |
| | | (SEQ ID NO: 10) |
| IL-6 | Forward | 5′-AGT-TGC-CTT-CTT-GGG-ACT-GA-3′ |
| | | (SEQ ID NO: 11) |
| | Reverse | 5′-CAG-AAT-TGC-CAT-TGC-ACA-AC-3′ |
| | | (SEQ ID NO: 12) |
| β-actin | Forward | 5′-GTT-GGG-ATG-AAC-CAG-AAG-GA-3′ |
| | | (SEQ ID NO: 13) |
| | Reverse | 5′-CTT-ACA-ATT-TCC-CGC-TCT-GC-3′ |
| | | (SEQ ID NO: 14) |

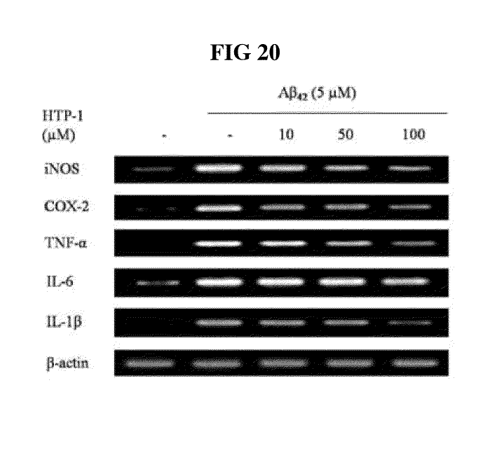

As a result, as illustrated in FIG. 20, it was confirmed that iNOS, COX-2, TNF-α, IL-6, and IL-1β mRNA expressions were increased by Aβ42 stimulation and for the treatment group treated with the HTP-1 peptide of the present invention, iNOS, COX-2, TNF-α, IL-6, and IL-1β mRNA expressions were effectively suppressed depending on the treated concentrations. Especially, it was confirmed that TNF-α and IL-1β mRNA expressions that are cytokines mediating the inflammation reaction were highly suppressed for the group treated with 100 μM of HTP-1. However, there was no significant change of IL-6 mRNA expression, which is the similar result to ELISA result in Example <2-5>.

<2-7> Effect of HTP-1 Peptide on Expressing Inflammation-Related Mediating Factors Protein According to a Pre-Treatment of HTP-1 Peptide of Present Invention for Activating BV2 Cells by Stimulating Aβ42

For a process of activating BV2 cells by stimulating Aβ42 in the experiment, effects of the HTP-1 peptide of the present invention on iNOS, COX-2, TNF-α, IL-6, and IL-1β protein expressions that are inflammation-related mediating factors were confirmed by using a western blot.

First, the HTP-1 peptides of the present invention with different concentrations were treated to BV2 cells. After 2 hours, Aβ42 (5) was treated and then cultured for 24 hours. Standard procedures were used for western blotting, where the whole cells were lysed in RIPA buffer. Cell debris was removed by centrifugation followed by quick freezing of the supernatants. The protein concentrations in the lysed cells were determined according to the Lowry method (BioRad Laboratories, Hercules, Calif.). The aliquot supernatant including the same amount of protein (15 μg) was electrophoresised on 10% or 12% SDS-PAGE gel and then transferred into a nitrocellulose membrane (Amersham Pharmacia Biotech., England, UK). The membrane was blocked in 5% skim milk dissolved in TBS including 0.1% tween 20 (TBS-T) for at least 1 hour and then was hybridized with a primary antibody (Santa Cruz Biotechnology Inc., CA, USA). All the primary monoclonal antibodies were diluted with TBS-T in a ratio of 1:1000. The membrane were blocked with 5% bovine serum albumin (BSA) and the incubated with different antibodies which were used to detect respective proteins using a chemiluminescent ECL assay kit, according to the manufacturer instructions. Western blots were visualized using a LAS3000® Luminescent image analyzer (Fujifilm Life Science, Tokyo, Japan).

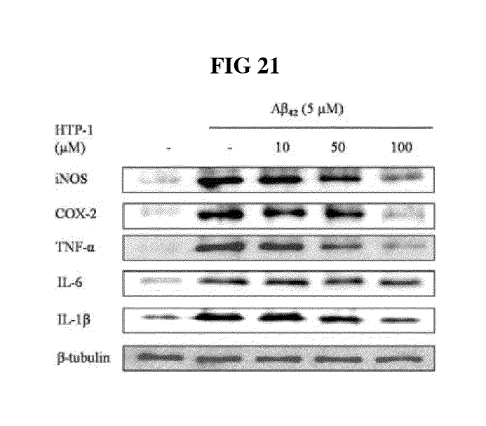

As a result, as illustrated in FIG. 21, it was confirmed that iNOS, COX-2, TNF-α, IL-6, and IL-1β protein expressions were increased by Aβ42 stimulation and for the treatment group treated with the HTP-1 peptide of the present invention, iNOS, COX-2, TNF-α, IL-6, and IL-1β protein expressions were effectively suppressed depending on the treated concentrations. However, there was no significant change of IL-6 protein expression, which is the similar result to the results in Examples <2-5> and <2-6>.

<2-8> Effect of HTP-1 Peptide on Expressing and Producing Cathepsin B and Cathepsin D According to a Pre-Treatment of HTP-1 Peptide of Present Invention for Activating BV2 Cells by Stimulating Aβ42

For a process of activating BV2 cells by stimulating Aβ42 in the experiment, effects of the HTP-1 peptide of the present invention on protease (involving in Aβ42 production process), cathepsin B and cathepsin D productions and expressions were confirmed.

Protease belonging to Cathepsin family is lysosome hydrolase that decomposes peptide and protein in lysosome under acidic pH. Cathepsins are divided into three sub-groups according to their active regions' amino acids; that is, cysteine cathepsin (B, C, H, F, K, L, O, S, V, and W), aspartate cathepsin (D and E), or serine cathepsin (G). Cathepsin D that is aspartate cathepsin has two aspartates resides in depth gap of active region. A main function of cathepsin D is catabolism in a cell in a lysosome section, but it is known that it involves in CNS disorder such as Alzheimer's disease (AD) and epilepsy (Dreyer et al., Eur J Biochem 1994, 224, 265-271; Hetman et al., Exp Neurol 1995, 136, 53-63). In addition, from the recent studies, it was found that in a case of blocking cathepsin B as protease in brain of Alzheimer's disease, a beta-amyloid protein was decreased in brain and also the memory was largely increased.

The effects of bioactive peptides on the cathepsin B and D levels were determined using enzyme immune assay. Briefly, the HTP-1 peptide of the present invention was pre-treated to BV2 cells. After 2 hours, Aβ42 (5) was added, and then cultured for 24 hours. In the cell culture supernatant, the cathepsin B and D levels were measured by using a commercial kit (Senso®lyte 440 cathepsin B assay kit and Senso®lyte 520 cathepsin B assay kit) according to the manufacturer's instructions.

As a result, as illustrated in FIG. 22, Aβ42-stimulated BV2 cells showed increase in cathepsin B and D productions. In contrast, pre-treatment of cells with HIP-1 resulted a significant reduction in cathepsin B and D productions in the presence of Aβ42. The reduction of cathepsin B releases varied dependently on HIP-1 concentration. Cathepsin B levels upon treatment with 10, 50, and 100 μM of HIP-1 were 94.93±2%, 68.46±5.51%, and 46.91±2.98%, respectively.

In addition, cathepsin B and D mRNA expressions were measured by RT-PCR and protein expressions were measured by western blot analysis. RT-PCR was performed by using the primer sequences in the following Table 4 and the same method as that of Example <2-6> and western blot was performed by using the same method as that of Example <2-7>.

TABLE 4
Primer sequence-2 for RT-PCR

| Gene | Primer sequence | |
|---|---|---|
| Cathepsin | Forward | 5′-CAC-AAC-TTC-TAC-AAC-GTG-G-3′ |
| B | | (SEQ ID NO: 15) |
| | Reverse | 5′-GTA-GAT-CTC-GGC-CAT-GAT-G-3′ |
| | | (SEQ ID NO: 16) |
| Cathepsin | Forward | 5′-CAA-CAG-AAG-CTG-GTG-GAC-AA-3′ |
| D | | (SEQ ID NO: 17) |
| | Reverse | 5′-AGA-TAC-GAC-AGC-ATT-GGC-A-3′ |
| | | (SEQ ID NO: 18) |
| β-actin | Forward | 5′-GTT-GGG-ATG-AAC-CAG-AAG-GA-3′ |
| | | (SEQ ID NO: 19) |
| | Reverse | 5′-CTT-ACA-ATT-TCC-CGC-TCT-GC-3′ |
| | | (SEQ ID NO: 20) |

As a result, as illustrated in FIG. 23, it was confirmed that cathepsin B and D mRNA expressions were increased by Aβ42 stimulation, and for the experimental group treated with the HIP-1 peptide of the present invention, cathepsin B and D mRNA and protein expressions were suppressed depending on the treated concentration. Especially, when cathepsin B expression was markedly increased with increasing the treated amount. In FIG. 23, (A) shows a result of RT-PCR and (B) shows a result of western blot analysis.

<2-9> Effect of HIP-1 Peptide on Producing and Expressing Anti-Inflammatory Cytokines According to a Pre-Treatment of HIP-1 Peptide of Present Invention for Activating BV2 Cells by Stimulating Aβ42

For a process of activating BV2 cells by stimulating Aβ42 in the experiment, effects of the HIP-1 peptide of the present invention on anti-inflammatory cytokines, such as IL-4, IL-13, and TNF-β productions and expressions were confirmed.

The levels of IL-4 and IL-13 productions were measured by using enzyme immune assay. Briefly, the HTP-1 peptides of the present invention with different concentrations (10, 50, 100 μM) were treated to BV2 cells. After 2 hours, Aβ42 (5) was treated and then cultured for 24 hours. The levels of IL-4 and IL-13 secretions in the cell culture supernatant were measured according to the manufacturer's instructions (R&D Systems Inc, Minneapolis, USA).

As a result, as illustrated in FIG. 24, it was confirmed that the level of IL-4 production was increased depending on the treated concentration in the group treated with the HTP-1 peptide of the present invention. However, the level of IL-13 production was rarely changed according to the treatment of the HTP-1 peptide of the present invention.

In addition, the levels of IL-4, IL-13, and TGF-β mRNA expressions were measured by using RT-PCR and the proteins expressions were measured by using a western blot analysis. The RT-PCR was performed by using the primer sequences in the following Table 5 and the same method as that of Example <2-6> and the western blot was performed by using the same method as that of Example <2-7>.

TABLE 5
Primer sequence-3 for RT-PCR

| Gene | Primer sequence | |
|---|---|---|
| TGF-β | Forward | 5′-CGA-GTG-CCA-AAT-GAA-GAG-GAC-C-3′ |
| | | (SEQ ID NO: 21) |
| | Reverse | 5′-AAA-CCT-GAG-CCA-GAA-CCT-GAC-G-3′ |
| | | (SEQ ID NO: 22) |
| IL-4 | Forward | 5′-GTA-CTG-TGC-AGC-CCT-GGA-AT-3′ |
| | | (SEQ ID NO: 23) |
| | Reverse | 5′-TTT-AGA-AAC-TGG-GCC-ACC-TC-3′ |
| | | (SEQ ID NO: 24) |
| IL-13 | Forward | 5′-GAA-TTT-GAG-CGT-CTC-TGT-CGA-A-3′ |
| | | (SEQ ID NO: 25) |
| | Reverse | 5′-GGT-TAT-GCC-AAA-TGC-ACT-TGA-G-3′ |
| | | (SEQ ID NO: 26) |
| β-actin | Forward | 5′-GTT-GGG-ATG-AAC-CAG-AAG-GA-3′ |
| | | (SEQ ID NO: 27) |
| | Reverse | 5′-CTT-ACA-ATT-TCC-CGC-TCT-GC-3′ |
| | | (SEQ ID NO: 28) |

As a result, as illustrated in FIG. 25, it was confirmed that for the group treated with the HTP-1 peptide of the present invention, the levels of TGF-β and IL-4 protein expressions were up-regulated depending the treated concentration. However, there were no changes in the levels of IL-13 mRNA and protein expressions according to the treatment of HTP-1 peptide of the present invention. In FIG. 25, (A) shows a result of RT-PCR and (B) shows a result of western blot.

As set forth above, according to exemplary embodiments of the invention, the bioactive peptide derived from seahorses according to the present invention can protect brain cells from a nerve toxin and oxidative stress, suppress expressions of inflammatory mediating factors, and increase expressions of anti-inflammatory cytokines, thereby having excellent anti-inflammatory activity, and also suppress expressions of cathepsin B and cathepsin D involving in a production process of Aβ42, a nerve toxin. Accordingly, the composition including the bioactive peptide as an effective component according to the present invention can exhibit excellent effects for preventing, or treating/improving a neurodegenerative disease through brain cells protecting activity, oxidative stress alleviating activity, anti-inflammatory activity, and protease inhibiting activity, thereby being usefully used as a functional medicine composition and food composition. Especially, the peptide of the present invention does not have toxicity, and is derived from seahorses that are used for drugs, thereby having stability without toxicity, so that the composition including the peptide as an effective component according to the present invention has stable advantage even if used for a long period time.

While the present invention has been shown and described in connection with the exemplary embodiments, it will be apparent to those skilled in the art that modifications and variations can be made without departing from the spirit and scope of the invention as defined by the appended claims.

What is claimed is:

1. A method of preventing or treating a neurodegenerative disease comprising administering to a subject in need thereof seahorse protein hydrolysates as an effective component.

2. The method of claim 1, wherein the seahorse is Hippocampus trimaculatus.

3. The method of claim 1, wherein the protein hydrolysates are one selected from the group of consisting of trypsin hydrolysates of the seahorses, alpha-chymotrypsin hydrolysates of the seahorses, papain hydrolysates of the seahorses, and protease E hydrolysates of the seahorses.

4. A method of preventing or treating a neurodegenerative disease comprising administering to a subject in need thereof the composition comprising a peptide having an amino acid sequence set forth in SEQ ID NO: 1 or SEQ ID NO: 2 as an effective component.

5. The method of claim 4, wherein the peptide is derived from Hippocampus trimaculatus.

6. The method of claim 4, wherein the peptide has an effect on preventing or treating a neurodegenerative disease through brain cells protecting activity, oxidative stress alleviating activity, anti-inflammatory activity, and protease inhibiting activity.

7. The method of claim 6, wherein the brain cells protecting activity is accomplished through a mechanism for inhibiting a neurotoxin release by activating microglia.

8. The method of claim 6, wherein the oxidative stress alleviating activity is accomplished through free radical scavenging activity.

9. The method of claim 6, wherein the anti-inflammatory activity is accomplished through inhibiting expressions of inflammation-related mediating factors such as NO, PGE2, iNOS, COX-2, TNF-α, and IL-1β and increasing expressions of anti-inflammatory cytokines such as TGF-β and IL-4.

10. The method of claim 6, wherein the protease inhibiting activity is accomplished through inhibiting expressions of cathepsin B and cathepsin D involving in a process of producing Aβ42.

11. The method of claim 4, wherein the peptide is included in a concentration of 10 to 10000 μM in a composition.

12. The method of claim 1, wherein the neurodegenerative disease is selected from the group consisting of Alzheimer's diseases (AD), Parkinson's diseases (PD), Lou Gehrig's diseases, Huntington's diseases (HD), amyotrophic lateral selerosis (ALS), multiple sclerosis, immune system-damaged brain function abnormalities, progressive nervous diseases, metabolic brain diseases, Niemann-Pick diseases, and dementias caused by ischemic stroke and cerebral hemorrhage.

13. The method of claim 4, wherein the neurodegenerative disease is selected from the group consisting of Alzheimer's diseases (AD), Parkinson's diseases (PD), Lou Gehrig's diseases, Huntington's diseases (HD), amyotrophic lateral selerosis (ALS), multiple sclerosis, immune system-damaged brain function abnormalities, progressive nervous diseases, metabolic brain diseases, Niemann-Pick diseases, and dementias caused by ischemic stroke and cerebral hemorrhage.
